(19) United States
(12) Patent Application Publication
(10) Pub. No.: US 2011/0021543 A1
(43) Pub. Date: Jan. 27, 2011

(54) HETEROCYCLIC COMPOUNDS AND THEIR USE AS PESTICIDES

(76) Inventors: Noelle Gauvry, Kembs-Loechle (FR); Jorg Fruchtel, Lorrach (DE); Thomas Goebel, Lorrach (DE); Sandra Schorderet Weber, Neuchatel (CH); Jacques Bouvier, Neuchatel (CH)

Correspondence Address: Novartis Animal Health US Inc., Suite 300, 3200 Northline Avenue, Greensboro, NC, 27408, US

(21) Appl. No.: 12/598,074

(22) PCT Filed: Apr. 29, 2008
(86) PCT No.: PCT/EP2008/055201

§ 371 Date: Mar. 23, 2010

(30) Foreign Application Priority Data
May 2, 2007 (EP) 07107323.3

(51) Int. Cl. A61K 31/437; C07D 471/04; C07D 495/04; A61K 31/407; C07D 491/048; C07D 209/52; A61K 31/403; C07D 487/04; A61K 31/519; A61P 33/14; A61P 33/00

(52) U.S. Cl. 514/265.1; 546/113; 514/300; 548/453; 514/421; 548/516; 514/412; 544/280

(57) ABSTRACT

Novel N-aryl substituted heteroindole compounds, processes for their manufacture, veterinary compositions containing such compounds, and methods of controlling ectoparasites are provided. The compounds are believed effective in the control of ectoparasites on warm-blooded productive livestock and domestic animals.

The present invention relates to new N-aryl substituted heteroindoles, processes for their manufacture, veterinary compositions containing said compounds and their use in the control of ectoparasites, especially ticks and fleas, on warm-blooded productive livestock and domestic animals.

There is an ongoing need for new active ingredients with improved pesticidal properties, for example, because currently used products cannot fulfil all the requirements concerning potency and activity spectrum. In addition, upcoming resistancy formation of ectoparasites against some known pesticides represent an issue. It has now surprisingly been found that new specific N-aryl substituted heteroindoles have excellent pesticidal properties, especially against ectoparasites.

The present invention therefore in one aspect relates to a compound of formula

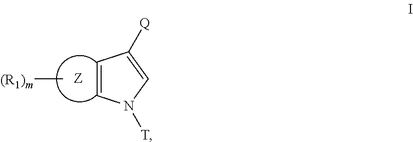

wherein
Z is an annulated carbocyclic or heterocyclic ring with the exception of a phenyl ring;
R₁ is halogen, cyano, nitro, C₁-C₆-alkyl, C₃-C₆-cycloalkyl, C₂-C₆-alkenyl, C₂-C₆-alkynyl, hydroxyl-C₁-C₆-alkyl, halo-C₁-C₆-alkyl, hydroxy, C₁-C₆-alkoxy, halo-C₁-C₆-alkoxy, SH, C₁-C₆-alkylthio, halo-C₁-C₆-alkylthio, C₁-C₆-alkylsulfinyl, halo-C₁-C₆-alkylsulfinyl, C₁-C₆-alkylsulfonyl, halo-C₁-C₆-alkylsulfonyl, SO₃R₃, SO₂NR₃R₄, NR₃R₄, COR₃, COOR₃ or CONR₃R₄, whereby, if m is greater than 1, the meanings of R₁ may be identical or different;
T is a group of the formula

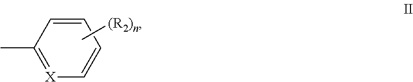

R₂ is halogen, cyano, nitro, C₁-C₆-alkyl, halo-C₁-C₆-alkyl, C₃-C₆-cycloalkyl, C₂-C₆-alkenyl, C₂-C₆-alkynyl, hydroxy, C₁-C₆-alkoxy, halo-C₁-C₆-alkoxy, SH, C₁-C₆-alkylthio, halo-C₁-C₆-alkylthio, C₁-C₆-alkylsulfinyl, halo-C₁-C₆-alkylsulfinyl, C₁-C₆-alkylsulfonyl, halo-C₁-C₆-alkylsulfonyl, SO₃R₃, SO₂NR₃R₄, NR₃R₄, COR₃, COOR₃, CONR₃R₄ or SF₅, whereby, if n is greater than 1, the meanings of R₂ may be identical or different;
R₃ and R₄ are independently from each other hydrogen, C₁-C₆-alkyl, which is unsubstituted or substituted by halogen, cyano, NO₂, C₁-C₄-alkoxy, C₁-C₄-alkylcarbonyl, C₁-C₄-alkylcarbonyloxy or C₁-C₄-alkoxycarbonyl, or is C₁-C₂-alkoxyC₁-C₂-alkyl;
m signifies 0, 1, 2 or 3;
n signifies 1, 2, 3 or 4;
X is N or C(R₂′), wherein R₂′ is hydrogen or has independently the meaning of R₂; with the proviso that R₂′ is not hydrogen if n is 1;
Q is a group of the formula

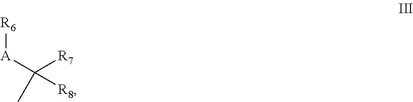

### A is O, S, S(O) or S(O₂);

R₆ is hydrogen, C₁-C₆-alkyl, C₁-C₆-hydroxyalkyl, C₃-C₆-cycloalkyl, C₃-C₆-cycloalkyl methyl, C₁-C₄-alkoxymethyl, C₁-C₂-alkoxy-C₁-C₂-alkoxymethyl, phenoxymethyl which is unsubstituted or substituted in the phenyl moiety by halogen, C₁-C₂-alkyl, halo-C₁-C₂-alkyl or C₁-C₂-alkoxy, benzyloxymethyl, C₂-C₆-alkenyl, C₂-C₆-alkynyl, halo-C₁-C₆-alkyl, halo-C₃-C₆-cycloalkyl, halo-C₁-C₆-cycloalkylmethyl, halo-C₂-C₆-alkenyl, halo-C₂-C₆-alkynyl, COR₃, COOR₃, CONR₃R₄, CSNR₃R₄, C₁-C₄-alkyl-silyl, phenyl or phenyl-C₁-C₂-alkyl, wherein the phenyl is each unsubstituted or substituted by halogen, nitro, cyano, hydroxy, C₁-C₄-alkyl, halo-C₁-C₄-alkyl, C₁-C₄-alkoxy, halo-C₁-C₄-alkoxy, NH₂, N—C₁-C₄-alkylamino, N,N-di-C₁-C₄-alkylamino, C₁-C₄-alkylthio, COR₃, COOR₃ or CONR₃R₄;
R₇ is C₁-C₆-alkyl, C₂-C₆-alkenyl, C₂-C₆-alkynyl, halo-C₁-C₆-alkyl, halo-C₂-C₆-alkenyl, halo-C₂-C₆-alkynyl, C₃-C₈-cycloalkyl, halo-C₃-C₈-cycloalkyl, hydroxy-C₁-C₆-alkyl, COR₃ or COOR₃; and
R₈ is hydrogen; C₁-C₆-alkyl which is unsubstituted or substituted by halogen, C₁-C₄-alkylthio, hydroxyl, C₁-C₄-alkoxy, amino or N-mono- or N,N-di-C₁-C₄-alkyl; unsubstituted or halogen-substituted C₂-C₆-alkenyl; unsubstituted or halogen-substituted C₂-C₆-alkynyl; unsubstituted or halogen-substituted C₃-C₈-cycloalkyl; C₅-C₆-cycloalkylmethyl wherein 1 to 3 carbon atoms of the cycloalkyl may be replaced by a heteroatom selected from the group consisting of NH, N(C₁-C₄-alkyl), O and S; benzyl; unsubstituted or halogen-, halo-C₁-C₂-alkyl- or halo-C₁-C₂-alkoxy-substituted phenyl; cyano, COR₃ or COOR₃;
or R₇ and R₈ together with the carbon atoms to which they are attached, form an aliphatic ring of 3 to 6 atoms, optionally including one additional heteroatom selected from the group consisting of nitrogen, sulfur or oxygen, or one carbonyl group, optionally substituted with 1 to 4 substituents, independently from each other selected from the group consisting of halogen, CN, NO₂, hydroxy, C₁-C₄-alkyl, and C₁-C₄-alkoxy.

The general terms used hereinbefore and hereinafter have the following meanings, unless defined otherwise.

Alkyl—as a group per se and as structural element of other groups and compounds, for example halogenalkyl, alkoxy, and alkylthio—is, in each case with due consideration of the specific number of carbon atoms in the group or compound in question, either straight-chained, i.e. methyl, ethyl, propyl, butyl, pentyl or hexyl, or branched, e.g. isopropyl, isobutyl, sec.-butyl, tert.-butyl, isopentyl, neopentyl or isohexyl, preferably straight-chained or branched C₁-C₄-alkyl and in particular C₁-C₂-alkyl.

Alkenyl—as a group per se and as structural element of other groups and compounds—is, in each case with due consideration of the specific number of carbon atoms in the group or compound in question and of the conjugated or isolated double bonds—either straight-chained, e.g. vinyl, allyl, 2-butenyl, 3-pentenyl, 1-hexenyl or 1,3-hexadienyl, or branched, e.g. isopropenyl, isobutenyl, isoprenyl, tert.-pentenyl or isohexenyl, preferably C₂-C₄-alkenyl and in particular vinyl or allyl.

Alkynyl—as a group per se and as structural element of other groups and compounds—is, in each case with due consideration of the specific number of carbon atoms in the group or compound in question and of the conjugated or isolated double bonds—either straight-chained, e.g. ethynyl, propargyl, 2-butinyl, 3-pentinyl, 1-hexinyl, 1-heptinyl or 3-hexen-1-inyl, or branched, e.g. 3-methylbut-1-inyl, 4-ethylpent-1-inyl or 4-methylhex-2-inyl, preferably C₂-C₄-alkynyl and in particular ethynyl.

Cycloalkyl—as a group per se and as structural element of other groups and compounds—is, in each case with due consideration of the specific number of carbon atoms in the group or compound in question, for example, cyclopropyl, cyclobutyl, cyclopentyl or cyclohexyl, in particular cyclopentyl or cyclohexyl.

Halogen—as a group per se and as structural element of other groups and compounds such as haloalkyl, haloalkoxy and haloalkylthio—is, for example, fluorine, chlorine, bromine or iodine, especially fluorine, chlorine or bromine, in particular fluorine or chlorine.

Halogen-substituted carbon-containing groups and compounds, such as haloalkyl, haloalk-oxy or haloalkylthio, may be partially halogenated or perhalogenated, whereby in the case of multiple halogenation, the halogen substituents may be identical or different. Examples of halogen-alkyl—as a group per se and as structural element of other groups and compounds such as halogen-alkoxy or halogen-alkylthio,—are methyl which is mono- to trisubstituted by fluorine, chlorine and/or bromine, such as CHF₂ or in particular CF₃; ethyl which is mono- to pentasubstituted by fluorine, chlorine and/or bromine, such as CH₂CF₃, CF₂CF₃, CF₂CCl₃, CF₂CHCl₂, CF₂CHF₂, CF₂CFCl₂, CF₂CHBr₂, CF₂CHClF, CF₂CHBrF or CClFCHClF; propyl or isopropyl, mono- to heptasubstituted by fluorine, chlorine and/or bromine, such as CH₂CHBrCH₂Br, CF₂CHFCF₃, CH₂CF₂CF₃ or CH(CF₃)₂; butyl or one of its isomers, mono- to nonasubstituted by fluorine, chlorine and/or bromine, such as CF(CF₃)CHFCF₃ or CH₂(CF₂)₂CF₃; pentyl or one of its isomers substituted once to eleven times by fluorine, chlorine and/or bromine, such as CF(CF₃)(CHF)₂CF₃ or CH₂(CF₂)₃CF₃; and hexyl or one of its isomers substituted once to thirteen times by fluorine, chlorine and/or bromine, such as (CH₂)₄CHBrCH₂Br, CF₂(CHF)₄CF₃, CH₂(CF₂)₄CF₃ or C(CF₃)₂(CHF)₂CF₃.

Alkoxy groups have a chain length of, for example, 1 to 6 carbon atoms, more preferably from 1 to 4 carbon atoms and in particular 1 or 2 carbon atoms. Alkoxy is for example methoxy, ethoxy, propoxy, isopropoxy, n-butoxy, isobutoxy, sec.-butoxy and tert.-butoxy, as well as the isomers pentyloxy and hexyloxy; preferably methoxy or ethoxy. Haloalkoxy groups preferably have a chain length of 1 to 6 carbon atoms. Haloalkoxy is e.g. fluoro-methoxy, difluoromethoxy, trifluoromethoxy, 2,2,2-trifluoroethoxy, 1,1,2,2-tetrafluoroethoxy, 2-fluoroethoxy, 2-chloroethoxy, 2,2-difluoroethoxy and 2,2,2-trichloroethoxy; preferably difluoromethoxy, 2-chloroethoxy or in particular trifluoromethoxy.

Alkylthio groups preferably have a chain length of 1 to 6 carbon atoms. Alkylthio is for example methylthio, ethylthio, propylthio, isopropylthio, n-butylthio, isobutylthio, sec.-butylthio or tert.-butylthio, preferably ethylthio or in particular methylthio.

Z is, for example, a 5- to 7-membered, preferably a 5- or 6-membered, carbocyclic or heterocyclic ring which is annulated in the 4- and 5-position of the pyrrol.

A suitable carbocyclic ring Z is, for example a 5- to 7-membered, preferably a 5- or 6-membered, cycloaliphatic ring, which may be substituted by 1 to 3 radicals R₁ as mentioned above. In particular, Z is a 5- or 6-membered, cycloaliphatic ring, which is further unsubstituted or substituted by a single radical R₁, that is m is preferably 0 or 1.

A suitable heterocyclic ring Z is, for example, a 5- or 6-membered ring having from 1 to 3, preferably 1 or 2, same or different heteroatoms selected from the group consisting of N, O and S, which ring may be substituted by 1 to 3 radicals R₁ as mentioned before. In particular, the heterocyclic ring Z is a 5- or 6-membered heteroaromatic ring having one heteroatom selected from the group consisting of N, O and S, which ring is further unsubstituted or substituted by a single substituent R₁, that is m is preferably 0 or 1.

A group of particularly preferred compounds according to the invention are thus those of formula

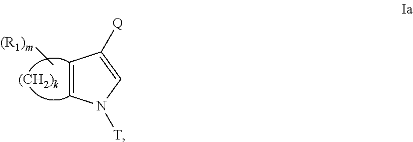

wherein k is the number 3, 4 or 5, in particular 3 or 4, and R₁, Q, T and m are as defined including the preferences given.

A further group of particularly preferred compounds according to the invention are thus those of formula

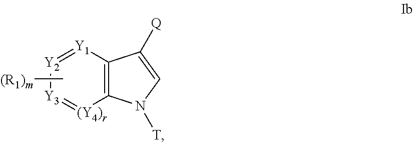

wherein 1 or 2 of the radicals Y₁, Y₂ Y₃ and Y₄ are independently a heteroatom selected from the group consisting of N, O and S and the remainder is CH, r is the number 0 or 1, and R₁, Q, T and m are as defined including the preferences given. In one embodiment of the compounds of formula Ib, r is 0, and 1 or 2, in particular 1, of the radicals Y₁, Y₂ and Y₃ is a heteroatom selected from the group consisting of N, O and S and the remainder is CH. In a further embodiment of the compounds of formula Ib, r is 1, and 1 or 2, in particular 1, of the radicals Y₁, Y₂ Y₃ and Y₄ is a heteroatom selected from the group consisting of N, O and S, in particular N, and the remainder is CH.

R₁ is preferably halogen, cyano, nitro, C₁-C₄-alkyl, hydroxyl-C₁-C₄-alkyl, halo-C₁-C₄-alkyl, hydroxy, C₁-C₄-alkoxy, halo-C₁-C₄-alkoxy, SH, C₁-C₄-alkylthio, halo-C₁-C₄-alkylthio, C₁-C₄-alkylsulfinyl, halo-C₁-C₄-alkylsulfinyl, C₁-C₄-alkylsulfonyl or halo-C₁-C₄-alkylsulfonyl; more preferably halogen, C₁-C₂-alkyl, halo-C₁-C₂-alkyl, C₁-C₂-alkoxy, halo-C₁-C₂-alkoxy, C₁-C₂-alkylthio or halo-C₁-C₂-alkylthio; and even more preferably halogen, C₁-C₂-alkyl or CF₃. In case m is greater than 1, the meanings of R₁ in each case may be identical or different.

The variable m in the compounds of formula I, Ia or Ib is preferably 0, 1 or 2, more preferably 0 or 1, and in particular 0.

In formula II, R₂ is preferably halogen, cyano, nitro, C₁-C₄-alkyl, halo-C₁-C₄-alkyl, C₂-C₄-alkenyl, C₂-C₄-alkynyl, hydroxy, C₁-C₄-alkoxy, halo-C₁-C₄-alkoxy, SH, C₁-C₄-alkylthio, halo-C₁-C₄-alkylthio or SF₅, more preferably halogen, cyano, nitro, halo-C₁-C₂-alkyl, C₂-C₄-alkenyl, C₂-C₄-alkynyl, halo-C₁-C₂-alkoxy, halo-C₁-C₂-alkylthio or SF₅, and most preferably halogen or halo-C₁-C₂-alkyl. In case n is greater than 1, the meanings of R₂ in each case may be identical or different, preferably different.

The variable n in the compounds of formula II is preferably 1 or 2, most preferably 2.

In formula II, X is preferably N or C(R₂′), wherein for R₂′ independently the meanings and preferences given above for R₂ apply. X is preferably a group C(R₂′), wherein R₂′ is halogen, in particular chlorine.

A particularly preferred radical T is of the formula

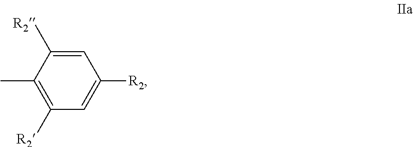

wherein R₂′ and R₂″ are each halogen, in particular chlorine, and R₂ is halo-C₁-C₂-alkyl, in particular CF₃.

In formula III, A is preferably O.

R₆ is preferably hydrogen, C₁-C₄-alkyl, halo-C₁-C₄-alkyl, hydroxy-C₁-C₄-alkyl, C₁-C₄-alkoxy-methyl, phenoxymethyl, benzyloxymethyl, phenyl, benzyl or COC₁-C₄-alkyl; more preferably hydrogen, C₁-C₂-alkyl, hydroxy-C₁-C₂-alkyl, C₁-C₂-alkoxymethyl or COC₁-C₂-alkyl; and in particular hydrogen, C₁-C₂-alkyl or CO—C₁-C₂-alkyl.

R₇ is preferably C₁-C₄-alkyl, halo-C₁-C₄-alkyl or hydroxy-C₁-C₄-alkyl; more preferably C₁-C₄-alkyl or halo-C₁-C₄-alkyl; even more preferably C₁-C₂-alkyl or halo-C₁-C₂-alkyl; and in particular CF₃.

R₈ is preferably hydrogen, C₁-C₄-alkyl which is unsubstituted or substituted by halogen, hydroxyl or C₁-C₂-alkoxy, C₂-C₄-alkenyl, C₂-C₄-alkynyl, benzyl, or unsubstituted or halogen-, halo-C₁-C₂-alkyl- or halo-C₁-C₂-alkoxy-substituted phenyl; more preferably hydrogen, C₁-C₄-alkyl, halo-C₁-C₄-alkyl, C₂-C₄-alkenyl or C₂-C₄-alkynyl; and in particular hydrogen, C₁-C₂-alkyl, CF₃, ethenyl or ethynyl.

If R₇ and R₈ together with the carbon atoms to which they are attached, form an aliphatic ring, this is preferably a piperidinyl or N—C₁-C₂-alkylpiperidinyl ring.

A preferred radical Q is of the above-given formula III, wherein A is O, R₆ is hydrogen, R₇ is CF₃, and for R₈ the above-given meanings and preferences apply.

A preferred embodiment of the invention concerns a compound of the above-given formula I, wherein R₁ is halogen or C₁-C₄-alkyl;

m signifies 0, 1 or 2;
T is a radical of formula II, wherein X is N or C(R₂′), n is 1 or 2, and R₂ and R₂′ are each independently halogen, cyano, nitro, C₂-C₄-alkynyl, halo-C₁-C₄-alkyl halo-C₁-C₄-alkoxy or SF₅, whereby, if n is 2, the meanings of R₂ may be identical or different;
Q is a group of formula III, wherein A is O, R₆ is hydrogen, C₁-C₄-alkyl or C(O)—C₁-C₂-alkyl, R₇ is C₁-C₄-alkyl or halo-C₁-C₄-alkyl, and R₈ is hydrogen; C₁-C₄-alkyl, halo-C₁-C₄-alkyl, C₂-C₄-alkenyl or C₂-C₄-alkynyl; and
for the ring Z the above given meanings and preferences apply.

A further preferred embodiment of the present invention concerns a compound of the formula

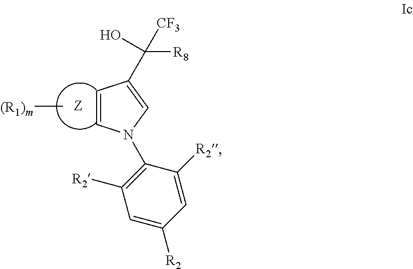

wherein for the ring Z, R₁, R₂, R₂′, R₈ and m each the above-given meanings and preferences apply, and R₂″ independently has the meaning of R₂. R₂′ in formula Ic is preferably halogen, in particular chlorine. Most preferably, R₂′ and R₂″ in formula Ic are each halogen, in particular chlorine, and R₂ is halo-C₁-C₂-alkyl, in particular CF₃.

A particularly preferred embodiment concerns a compound of formula Ic above, wherein

R₁ is halogen or C₁-C₂-alkyl;
m is 0 or 1, in particular 0;
R₂ is trifluoromethyl, and R₂′ and R₂″ are each chlorine;
R₈ is C₁-C₂-alkyl, halo-C₁-C₂-alkyl, C₂-C₄-alkenyl or C₂-C₄-alkynyl: and
the ring Z is a 5- or 6-membered cycloaliphatic ring or a 5- or 6-membered heteroaromatic ring having one heteroatom selected from the group consisting of N, O and S.

The compounds of the formula I according to the present invention may be prepared, for example, by a process, which comprises

(i) halogenating a compound of formula

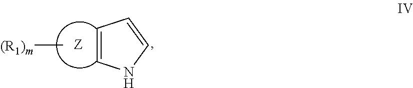

wherein R₁, Z and m are defined as given above, to yield a compound of the formula

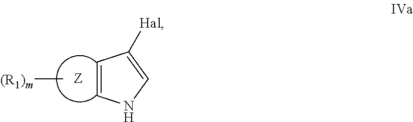

wherein Hal is halogen, for example bromine, and R₁, Z and m are defined as given above;
(ii) reacting the compound of the formula IVa obtained according to step (i) with a compound of the formula

L-T V,

wherein T is defined as given above and L is a leaving group, optionally in the presence of a basic catalyst, to yield a compound of the formula

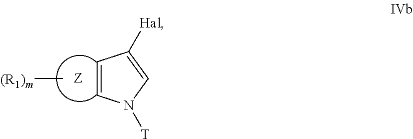

wherein Hal is halogen, for example bromine, and R₁, T, Z and m are defined as given above; and
(iii) reacting the compound of the formula IVb with a lithium-organic compound, for example with n-butyllithium, followed by reacting the resulting lithium-organic compound with a ketone of the formula

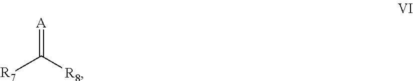

wherein A, R₇ and R₈ are as defined, to yield a compound of the formula I.

The compounds of formula IV may be halogenated in step (i) in a manner known per se from organic textbooks. For example bromination, of a compound of formula IV, may be performed with bromine or N-bromosuccinimide (NBS).

The reaction partners in step (ii) can be reacted with one another as they are, i.e. without the addition of a solvent or diluent, e.g. in the melt. In most cases, however, the addition of an inert solvent or diluent, or a mixture thereof, is of advantage. Examples of such solvents or diluents are: aromatic, aliphatic and alicyclic hydrocarbons and halogenated hydrocarbons, such as benzene, toluene, xylene, mesitylene, tetraline, chlorobenzene, dichlorobenzene, bromobenzene, petroleum ether, hexane, cyclohexane, dichloromethane, trichloromethane, tetrachloromethane, dichloroethane, trichloroethene or tetrachloroethene; ethers, such as diethyl ether, dipropyl ether, diisopropyl ether, dibutyl ether, tert-butyl methyl ether, ethylene glycol monomethyl ether, ethylene glycol monoethyl ether, ethylene glycol dimethylether, dimethoxydiethylether, tetrahydrofuran or dioxane; ketones such as acetone, methyl ethyl ketone or methyl isobutyl ketone; amides such as N,N-dimethylformamide, N,N-diethyl-formamide, N,N-dimethylacetamide, N-methylpyrrolidone or hexamethylphosphoric acid triamide; nitriles such as acetonitrile or propionitrile; and sulfoxides, such as dimethyl sulfoxide.

Suitable bases for facilitating the reaction are e.g. alkali metal or alkaline earth metal hydroxides, hydrides, amides, alkanolates, acetates, carbonates, dialkylamides or alkylsilyl-amides; alkylamines, alkylenediamines, optionally N-alkylated, optionally unsaturated, cyclo-alkylamines, basic heterocycles, ammonium hydroxides, as well as carbocyclic amines. Those which may be mentioned by way of example are sodium hydroxide, hydride, amide, methanolate, acetate, carbonate, potassium tert.-butanolate, hydroxide, carbonate, hydride, lithium diisopropylamide, potassium bis(trimethylsilyl)-amide, calcium hydride, triethylamine, diisopropylethylamine, triethylenediamine, cyclohexylamine, N-cyclohexyl-N,N-dimethyl-amine, N,N-diethylaniline, pyridine, 4-(N,N-dimethylamino)pyridine, quinuclidine, N-methyl-morpholine, benzyltrimethylammonium hydroxide, as well as 1,5-diazabicyclo[5.4.0]undec-5-ene (DBU).

A preferred leaving group L is halogen, especially fluorine or chlorine.

The reaction advantageously takes place in a temperature range of ca. 0° C. to ca. 150° C., preferably from ca. 50° C. to ca. 120° C.

In a preferred process, a compound of formula IVa is reacted at 90° C. in an amide, preferably N,N-dimethylformamide, with a compound of formula V in the presence of a base, preferably potassium carbonate.

The compounds of the formula IVa and V are known and commercially available, or may be prepared according to methods well known in the art, for example, from textbooks of organic chemistry.

In step (iii), the metalation and the further reaction with the compounds of the formula VI may all be performed in a manner known per se from textbooks of organic chemistry.

An alternative process for the manufacture of the compounds of the formula I comprises the steps of

(i) reacting a compound of the above-given formula IV with a carboxylic acid halide or anhydride of the formula

R₇—C(O)-Hal VIa

or

R₇—C(O)—O—C(O)—R₇ VIb

to yield a compound of the formula

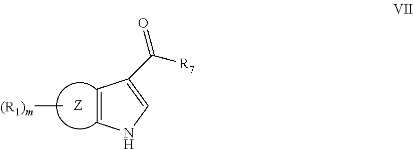

wherein Z, R₁, R₇ and m are as defined,
(ii) reacting the compound of the formula VII obtained according to step (i) with a compound of formula

L-T V

wherein T is defined as given above and L is a leaving group, optionally in the presence of a basic catalyst, to yield a compound of the formula

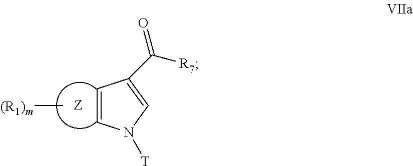

and
(iii) reacting the compound of the formula VIIa obtained according to step (ii) with a compound of the formula

R₈−L₁ VIII,

wherein L₁ is a leaving group and R₈ is as defined above, to yield a compound of the formula I.

In step (iii), the reaction partners can be reacted with one another as they are, i.e. without the addition of a solvent or diluent, e.g. in the melt. In most cases, however, the addition of an inert solvent or diluent, or a mixture thereof, is of advantage. Examples of such solvents or diluents are: aromatic, aliphatic and alicyclic hydrocarbons and halogenated hydrocarbons, such as benzene, toluene, xylene, mesitylene, tetraline, chlorobenzene, dichlorobenzene, bromobenzene, petroleum ether, hexane, cyclohexane, dichloromethane, trichloromethane, tetrachloromethane, dichloroethane, trichloroethene or tetrachloroethene; ethers, such as diethyl ether, dipropyl ether, diisopropyl ether, dibutyl ether, tert-butyl methyl ether, ethylene glycol monomethyl ether, ethylene glycol monoethyl ether, ethylene glycol dimethylether, dimethoxydiethylether, tetrahydrofuran or dioxane; amides such as N,N-dimethylformamide, N,N-diethylformamide, N,N-dimethylacetamide, N-methylpyrrolidone or hexamethyl-phosphoric acid triamide; nitriles such as acetonitrile or propionitrile; and sulfoxides, such as dimethyl sulfoxide.

Preferred leaving groups L₁ are MgBr, MgCl, Mgl or Li, especially MgBr.

The reaction advantageously takes place in a temperature range of ca. −20° C. to ca. 100° C., preferably from ca. 0° C. to ca. 30° C.

In a preferred process, a compound of formula VIIa is reacted at room temperature in an ether, preferably diethyl ether, with a compound of formula VIII.

A compound of formula I obtained according to an above-described process may also be converted to another compound of formula I by reactions known per se from textbooks of organic chemistry. For example, a compound of formula I, wherein Q is a radical —C(OH)(R₇)(R₈) may easily be converted to a compound of formula I with a different radical —C(OR₆)(R₇)(R₈) by a suitable acylation or etherification reaction; or a compound of formula I wherein Q is a radical —C(O)—R₅ may be converted by reductive alkylation to a compound wherein Q is a radical —C(OH)(R₇)(R₈).

Salts of compounds I may be produced in known manner. Acid addition salts, for example, are obtainable from compounds I by treating with a suitable acid or a suitable ion exchange reagent, and salts with bases are obtainable by treating with a suitable base or a suitable ion exchange reagent.

Salts of compounds I can be converted into the free compounds I by the usual means, acid addition salts e.g. by treating with a suitable basic composition or with a suitable ion exchange reagent, and salts with bases e.g. by treating with a suitable acid or a suitable ion exchange reagent.

Salts of compounds I can be converted into other salts of compounds I in a known manner; acid addition salts can be converted for example into other acid addition salts, e.g. by treating a salt of an inorganic acid, such as a hydrochloride, with a suitable metal salt, such as a sodium, barium, or silver salt, of an acid, e.g. with silver acetate, in a suitable solvent, in which a resulting inorganic salt, e.g. silver chloride, is insoluble and thus precipitates out from the reaction mixture.

Depending on the method and/or reaction conditions, the compounds of formula I with salt-forming characteristics can be obtained in free form or in the form of salts.

The compounds of formula I can also be obtained in the form of their hydrates and/or also can include other solvents, used for example where necessary for the crystallisation of compounds present in solid form.

The compounds of the formula I or Ia may be optionally present as optical and/or geometric isomers or as a mixture thereof. The invention relates both to the pure isomers and to all possible isomeric mixtures, and is hereinbefore and hereinafter understood as doing so, even if stereochemical details are not specifically mentioned in every case.

Diastereoisomeric mixtures of compounds of formula I and Ia, which are obtainable by the process or in another way, may be separated in known manner, on the basis of the physical-chemical differences in their components, into the pure diastereoisomers, for example by fractional crystallisation, distillation and/or chromatography.

Splitting of mixtures of enantiomers, that are obtainable accordingly, into the pure isomers, may be achieved by known methods, for example by recrystallisation from an optically active solvent, by chromatography on chiral adsorbents, e.g. high-pressure liquid chromatography (HPLC) on acetyl cellulose, with the assistance of appropriate micro-organisms, by cleavage with specific immobilised enzymes, through the formation of inclusion compounds, e.g. using chiral crown ethers, whereby only one enantiomer is complexed.

According to the invention, apart from separation of corresponding isomer mixtures, generally known methods of diastereoselective or enantioselective synthesis can also be applied to obtain pure diastereoisomers or enantiomers, e.g. by carrying out the method of the invention using educts with correspondingly suitable stereochemistry.

It is advantageous to isolate or synthesise the biologically more active isomer, e.g. enantiomer, provided that the individual components have differing biological efficacy. In the method of the present invention, the starting materials and intermediates used are preferably those that lead to the compounds I described at the beginning as being especially useful.

The invention relates especially to the method of preparation described in the example. Starting materials and intermediates, which are new and are used according to the invention for the preparation of the compounds of formula I, as well as their usage and process for the preparation thereof, similarly form an object of the invention.

The compounds of formula I or Ia according to the invention are notable for their broad activity spectrum and are valuable active ingredients for use in pest control, including in particular the control of endo- and ecto-parasites in and on animals, whilst being well-tolerated by warm-blooded animals, fish and plants.

In the context of the present invention, ectoparasites are understood to be in particular insects, acari (mites and ticks), and crustaceans (sea lice). These include insects of the following orders: Lepidoptera, Coleoptera, Homoptera, Hemiptera, Heteroptera, Diptera, Dictyoptera, Thysanoptera, Orthoptera, Anoplura, Siphonaptera, Mallophaga, Thysanura, Isoptera, Psocoptera and Hymenoptera. However, the ectoparasites which may be mentioned in particular are those which trouble humans or animals and carry pathogens, for example flies such as Musca domestica, Musca vetustissima, Musca autumnalis, Fannia canicularis, Sarcophaga carnaria, Lucilia cuprina, Lucilia sericata, Hypoderma bovis, Hypoderma lineatum, Chrysomyia chloropyga, Dermatobia hominis, Cochliomyia hominivorax, Gasterophilus intestinalis, Oestrus ovis, biting flies such as Haematobia irritans irritans, Haematobia irritans exigua, Stomoxys calcitrans, horse-flies (Tabanids) with the subfamilies of Tabanidae such as Haematopota spp. (e.g. Haematopota pluvialis) and Tabanus spp, (e.g. Tabanus nigrovittatus) and Chrysopsinae such as Chrysops spp. (e.g. Chrysops caecutiens); Hippoboscids such as Melophagus ovinus (sheep ked); tsetse flies, such as Glossinia spp. other biting insects like midges, such as Ceratopogonidae (biting midges), Simuliidae (Blackflies), Psychodidae (Sandflies); but also blood-sucking insects, for example mosquitoes, such as Anopheles spp, Aedes spp and Culex spp, fleas, such as Ctenocephalides felis and Ctenocephalides canis (cat and dog fleas), Xenopsylla cheopis, Pulex irritans, Ceratophyllus gallinae, Dermatophilus penetrans, blood-sucking lice (Anoplura) such as Linognathus spp, Haematopinus spp, Solenopotes spp, Pediculus humanis; but also chewing lice (Mallophaga) such as Bovicola (Damalinia) ovis, Bovicola (Damalinia) bovis and other Bovicola spp. Ectoparasites also include members of the order Acarina, such as mites (e.g. Chorioptes bovis, Cheyletiella spp., Dermanyssus gallinae, Demodex canis, Sarcoptes scabiei, Psoroptes ovis and Psorergates spp. and ticks. Known representatives of ticks are, for example, Boophilus, Amblyomma, Anocentor, Dermacentor, Haemaphysalis, Hyalomma, Ixodes, Rhipicentor, Margaropus, Rhipicephalus, Argas, Otobius and Ornithodoros and the like, which preferably infest warm-blooded animals including farm animals, such as cattle, horses, pigs, sheep and goats, poultry such as chickens, turkeys, guineafowls and geese, fur-bearing animals such as mink, foxes, chinchillas, rabbits and the like, as well as domestic animals such as cats and dogs, but also humans.

Compounds of formula I can also be used against hygiene pests, especially of the order Diptera of the families Muscidae, Sarcophagidae, Anophilidae and Culicidae; the orders Orthoptera, Dictyoptera (e.g. the family Blattidae (cockroaches), such as Blatella germanica, Blatta orientalis, Periplaneta americana) and Hymenoptera(e.g. the families Formicidae (ants) and Vespidae (wasps).

Compounds of formula I also have sustainable efficacy on parasitic mites and insects of plants. In the case of spider mites of the order Acarina, they are effective against eggs, nymphs and adults of Tetranychidae (Tetranychus spp. and Panonychus spp.).

They have high activity against sucking insects of the order Homoptera, especially against pests of the families Aphididae, Delphacidae, Cicadellidae, Psyllidae, Loccidae, Diaspididae and Eriophydidae (e.g. rust mite on citrus fruits); the orders Hemiptera, Heteroptera and Thysanoptera, and on the plant-eating insects of the orders Lepidoptera, Coleoptera, Diptera and Orthoptera

They are similarly suitable as a soil insecticide against pests in the soil.

The compounds of formula I are therefore effective against all stages of development of sucking insects and eating insects on crops such as cereals, cotton, rice, maize, soya, potatoes, vegetables, fruit, tobacco, hops, citrus, avocados and other crops.

The compounds of formula I are also effective against plant nematodes of the species Meloidogyne, Heterodera, Pratylenchus, Ditylenchus, Radopholus, Rizoglyphus etc.

In particular, the compounds are effective against helminths, in which the endoparasitic nematodes and trematodes may be the cause of serious diseases of mammals and poultry, e.g. sheep, pigs, goats, cattle, horses, donkeys, dogs, cats, guinea-pigs and exotic birds. Typical nematodes of this indication are: Haemonchus, Trichostrongylus, Ostertagia, Nematodirus, Cooperia, Ascaris, Bunostonum, Oesophagostonum, Charbertia, Trichuris, Strongylus, Trichonema, Dictyocaulus, Capillaria, Heterakis, Toxocara, Ascaridia, Oxyuris, Ancylostoma, Uncinaria, Toxascaris and Parascaris. The trematodes include Clonorchis, Dicrocoelium, Echinostoma and in particular, the family of Fasciolideae, especially Fasciola hepatica. The particular advantage of the compounds of formula I is their efficacy against those parasites that are resistant towards active ingredients based on benzimidazoles.

Certain pests of the species Nematodirus, Cooperia and Oesophagostonum infest the intestinal tract of the host animal, while others of the species Haemonchus and Ostertagia are parasitic in the stomach and those of the species Dictyocaulus are parasitic in the lung tissue. Parasites of the families Filariidae and Setariidae may be found in the internal cell tissue and in the organs, e.g. the heart, the blood vessels, the lymph vessels and the subcutaneous tissue. A particularly notable parasite is the heartworm of the dog, Dirofilaria immitis. The compounds of formula I are highly effective against these parasites.

Furthermore, the compounds of formula I are suitable for the control of human pathogenic parasites. Of these, typical representatives that appear in the digestive tract are those of the species Ancylostoma, Necator, Ascaris, Strongyloides, Trichinella, Capillaria, Trichuris and Enterobius. The compounds of the present invention are also effective against parasites of the species Wuchereria, Brugia, Onchocerca and Loa from the family of Filariidae, which appear in the blood, in the tissue and in various organs, and also against Dracunculus and parasites of the species Strongyloides and Trichinella, which infect the gastrointestinal tract in particular.

The good pesticidal activity of the compounds of formula I according to the invention corresponds to a mortality rate of at least 50-60% of the pests mentioned. In particular, the compounds of formula I are notable for the exceptionally long duration of efficacy. The compounds of formula I are preferably employed in unmodified form or preferably together with the adjuvants conventionally used in the art of formulation and may therefore be processed in a known manner to give, for example, emulsifiable concentrates, directly dilutable solutions, dilute emulsions, soluble powders, granules or micro-encapsulations in polymeric substances. As with the compositions, the methods of application are selected in accordance with the intended objectives and the prevailing circumstances.

The formulation, i.e. the agents, preparations or compositions containing the active ingredient of formula I, or combinations of these active ingredients with other active ingredients, and optionally a solid or liquid adjuvant, are produced in a manner known per se, for example by intimately mixing and/or grinding the active ingredients with spreading compositions, for example with solvents, solid carriers, and optionally surface-active compounds (surfactants).

The solvents in question may be: alcohols, such as ethanol, propanol or butanol, and glycols and their ethers and esters, such as propylene glycol, dipropylene glycol ether, ethylene glycol, ethylene glycol monomethyl or -ethyl ether, ketones, such as cyclohexanone, isophorone or diacetanol alcohol, strong polar solvents, such as N-methyl-2-pyrrolidone, dimethyl sulfoxide or N,N-dimethylformamide, or water, vegetable oils, such as rape, castor, coconut, or soybean oil, and also, if appropriate, silicone oils. Preferred application forms for usage on warm-blooded animals in the control of helminths include solutions, emulsions, suspensions (drenches), food additives, powders, tablets including effervescent tablets, boli, capsules, micro-capsules and pour-on formulations, whereby the physiological compatibility of the formulation excipients must be taken into consideration.

The binders for tablets and boli may be chemically modified polymeric natural substances that are soluble in water or in alcohol, such as starch, cellulose or protein derivatives (e.g. methyl cellulose, carboxymethyl cellulose, ethylhydroxyethyl cellulose, proteins such as zein, gelatine and the like), as well as synthetic polymers, such as polyvinyl alcohol, polyvinyl pyrrolidone etc. The tablets also contain fillers (e.g. starch, microcrystalline cellulose, sugar, lactose etc.), glidants and disintegrants.

If the anthelminthics are present in the form of feed concentrates, then the carriers used are e.g. performance feeds, feed grain or protein concentrates. Such feed concentrates or compositions may contain, apart from the active ingredients, also additives, vitamins, antibiotics, chemotherapeutics or other pesticides, primarily bacteriostats, fungistats, coccidiostats, or even hormone preparations, substances having anabolic action or substances which promote growth, which affect the quality of meat of animals for slaughter or which are beneficial to the organism in another way. If the compositions or the active ingredients of formula I contained therein are added directly to feed or to the drinking troughs, then the formulated feed or drink contains the active ingredients preferably in a concentration of ca. 0.0005 to 0.02% by weight (5-200 ppm).

The compounds of formula I according to the invention may be used alone or in combination with other biocides. They may be combined with pesticides having the same sphere of activity e.g. to increase activity, or with substances having another sphere of activity e.g. to broaden the range of activity. It can also be sensible to add so-called repellents. If the range of activity is to be extended to endoparasites, e.g. wormers, the compounds of formula I are suitably combined with substances having endoparasitic properties. Of course, they can also be used in combination with antibacterial compositions. Since the compounds of formula I are adulticides, i.e. since they are effective in particular against the adult stage of the target parasites, the addition of pesticides which instead attack the juvenile stages of the parasites may be very advantageous. In this way, the greatest part of those parasites that produce great economic damage will be covered. Moreover, this action will contribute substantially to avoiding the formation of resistance. Many combinations may also lead to synergistic effects, i.e. the total amount of active ingredient can be reduced, which is desirable from an ecological point of view. Preferred groups of combination partners and especially preferred combination partners are named in the following, whereby combinations may contain one or more of these partners in addition to a compound of formula I.

Suitable partners in the mixture may be biocides, e.g. the insecticides and acaricides with a varying mechanism of activity, which are named in the following and have been known to the person skilled in the art for a long time, e.g. chitin synthesis inhibitors, growth regulators; active ingredients which act as juvenile hormones; active ingredients which act as adulticides; broad-band insecticides, broad-band acaricides and nematicides; and also the well known anthelminthics and insect- and/or acarid-deterring substances, said repellents or detachers.

Non-limitative examples of suitable insecticides and acaricides are disclosed, for example, in WO 2005/058802 on pages 13-15, Nrs. 1. to 185. Non-limitative examples of suitable anthelminthics are named, for example, in WO 2005/058802 on page 16, Nrs. (A1) to (A12). Non-limitative examples of suitable repellents and detachers are: (i) DEET (N,N-diethyl-m-toluamide), (ii) KBR 3023 N-butyl-2-oxycarbonyl-(2-hydroxy)-piperidine, and (iii) Cymiazole=N,-2,3-dihydro-3-methyl-1,3-thiazol-2-ylidene-2,4-xylidene.

The said partners in the mixture are best known to specialists in this field. Most are described in various editions of the Pesticide Manual, The British Crop Protection Council, London, and others in the various editions of The Merck Index, Merck & Co., Inc., Rahway, N.J., USA or in patent literature. A list of suitable partners including a reference is disclosed in WO 2005/058802 on pages 16-21, No. (I) to (CLXXXIII).

As a consequence of the above details, a further essential aspect of the present invention relates to combination preparations for the control of parasites on warm-blooded animals, characterised in that they contain, in addition to a compound of formula I, at least one further active ingredient having the same or different sphere of activity and at least one physiologically acceptable carrier. The present invention is not restricted to two-fold combinations.

As a rule, the compositions according to the invention contain 0.1 to 99% by weight, especially 0.1 to 95% by weight of active ingredient of formula I, Ia or mixtures thereof, 99.9 to 1% by weight, especially 99.8 to 5% by weight of a solid or liquid admixture, including 0 to 25% by weight, especially 0.1 to 25% by weight of a surfactant.

Application of the compositions according to the invention to the animals to be treated may take place topically, perorally, parenterally or subcutaneously, the composition being present in the form of solutions, emulsions, suspensions, (drenches), powders, tablets, boli, capsules and pour-on formulations.

The pour-on or spot-on method consists in applying the compound of formula I to a specific location of the skin or coat, advantageously to the neck or backbone of the animal. This takes place e.g. by applying a swab or spray of the pour-on or spot-on formulation to a relatively small area of the coat, from where the active substance is dispersed almost automatically over wide areas of the fur owing to the spreading nature of the components in the formulation and assisted by the animal's movements.

Pour-on or spot-on formulations suitably contain carriers, which promote rapid dispersement over the skin surface or in the coat of the host animal, and are generally regarded as spreading oils. Suitable carriers are e.g. oily solutions; alcoholic and isopropanolic solutions such as solutions of 2-octyldodecanol or oleyl alcohol; solutions in esters of monocarboxylic acids, such as isopropyl myristate, isopropyl palmitate, lauric acid oxalate, oleic acid oleyl ester, oleic acid decyl ester, hexyl laurate, oleyl oleate, decyl oleate, capric acid esters of saturated fat alcohols of chain length C₁₂-C₁₈; solutions of esters of dicarboxylic acids, such as dibutyl phthalate, diisopropyl isophthalate, adipic acid diisopropyl ester, di-n-butyl adipate or also solutions of esters of aliphatic acids, e.g. glycols. It may be advantageous for a dispersing agent to be additionally present, such as one known from the pharmaceutical or cosmetic industry. Examples are 2-pyrrolidone, 2-(N-alkyl)pyrrolidone, acetone, polyethylene glycol and the ethers and esters thereof, propylene glycol or synthetic triglycerides. The oily solutions include e.g. vegetable oils such as olive oil, groundnut oil, sesame oil, pine oil, linseed oil or castor oil. The vegetable oils may also be present in epoxidised form. Paraffins and silicone oils may also be used.

A pour-on or spot-on formulation generally contains 1 to 20% by weight of a compound of formula I, 0.1 to 50% by weight of dispersing agent and 45 to 98.9% by weight of solvent. The pour-on or spot-on method is especially advantageous for use on herd animals such as cattle, horses, sheep or pigs, in which it is difficult or time-consuming to treat all the animals orally or by injection. Because of its simplicity, this method can of course also be used for all other animals, including individual domestic animals or pets, and is greatly favoured by the keepers of the animals, as it can often be carried out without the specialist presence of the veterinarian.

Whereas it is preferred to formulate commercial products as concentrates, the end user will normally use dilute formulations.

Such compositions may also contain further additives, such as stabilisers, anti-foaming agents, viscosity regulators, binding agents or tackifiers, as well as other active ingredients, in order to achieve special effects.

Compositions of this type, which are used by the end user, similarly form a constituent of the present invention.

In each of the processes according to the invention for pest control or in each of the pest control compositions according to the invention, the active ingredients of formula I can be used in all of their steric configurations or in mixtures thereof.

The invention also includes a method of prophylactically protecting warm-blooded animals, especially productive livestock, domestic animals and pets, against parasitic pests, which is characterised in that the active ingredients of formula or the active ingredient formulations prepared therefrom are administered to the animals as an additive to the feed, or to the drinks or also in solid or liquid form, orally or by injection or parenterally. The invention also includes the compounds of formula I according to the invention for usage in one of the said processes.

Preferred formulations of the compounds of the invention are made up as follows:

(%=percent by weight)

### Formulation Examples

### 1. Granulate

| | a) | b) |
|---|---|---|
| active ingredient | 5% | 10% |
| kaolin | 94% | — |
| highly dispersed silicic acid | 1% | — |
| attapulgite | — | 90% |

The active ingredient is dissolved in methylene chloride, sprayed onto the carrier and the solvent subsequently concentrated by evaporation under vacuum. Granulates of this kind can be mixed with the animal feed.

### 2. Granulate

| | |
|---|---|
| active ingredient | 3% |
| polyethylene glycol (mw 200) | 3% |
| kaolin | 94% |

(mw = molecular weight)

The finely ground active ingredient is evenly applied in a mixer to the kaolin which has been moistened with polyethylene glycol. In this way, dust-free coated granules are obtained.

### 3. Tablets or Boli

| | | |
|---|---|---|
| I | active ingredient | 33.00% |
| | methylcellulose | 0.80% |
| | silicic acid, highly dispersed | 0.80% |
| | corn starch | 8.40% |
| II | lactose, cryst. | 22.50% |
| | corn starch | 17.00% |
| | microcryst. cellulose | 16.50% |
| | magnesium stearate | 1.00% |

I Methyl cellulose is stirred into water. After the material has swollen, silicic acid is stirred in and the mixture homogeneously suspended. The active ingredient and the corn starch are mixed. The aqueous suspension is worked into this mixture and kneaded to a dough. The resulting mass is granulated through a 12 M sieve and dried.
II All 4 excipients are mixed thoroughly.
III The preliminary mixes obtained according to I and II are mixed and pressed into tablets or boli.

### 4. Injectables

A. Oily Vehicle (Slow Release)

| | | | |
|---|---|---|---|
| 1. | active ingredient | 0.1-1.0 | g |
| | groundnut oil | ad 100 | ml |
| 2. | active ingredient | 0.1-1.0 | g |
| | sesame oil | ad 100 | ml |

Preparation: The active ingredient is dissolved in part of the oil whilst stirring and, if required, with gentle heating, then after cooling made up to the desired volume and sterile-filtered through a suitable membrane filter with a pore size of 0.22 mm.

B. Water-Miscible Solvent (Average Rate of Release)

| | | |
|---|---|---|
| active ingredient | 0.1-1.0 | g |
| 4-hydroxymethyl-1,3-dioxolane (glycerol formal) | 40 | g |
| 1,2-propanediol | ad 100 | ml |
| active ingredient | 0.1-1.0 | g |
| glycerol dimethyl ketal | 40 | g |
| 1,2-propanediol | ad 100 | ml |

Preparation: The active ingredient is dissolved in part of the solvent whilst stirring, made up to the desired volume and sterile-filtered through a suitable membrane filter with a pore size of 0.22 mm.

C. Aqueous Solubilisate (Rapid Release)

| | | | |
|---|---|---|---|
| 1. | active ingredient | 0.1-1.0 | g |
| | polyethoxylated castor oil (40 ethylene oxide units) | 10 | g |
| | 1,2-propanediol | 20 | g |
| | benzyl alcohol | 1 | g |
| | aqua ad inject. | ad 100 | ml |
| 2. | active ingredient | 0.1-1.0 | g |
| | polyethoxylated sorbitan monooleate (20 ethylene | 8 | g |
| | oxide units) | | |
| | 4-hydroxymethyl-1,3-dioxolane (glycerol formal) | 20 | g |
| | benzyl alcohol | 1 | g |
| | aqua ad inject. | ad 100 | ml |

Preparation: The active ingredient is dissolved in the solvents and the surfactant, and made up with water to the desired volume. Sterile filtration through an appropriate membrane filter of 0.22 mm pore size.

### 5. Pour on

A.

| | | |
|---|---|---|
| active ingredient | 5 | g |
| isopropyl myristate | 10 | g |
| isopropanol | ad 100 | ml |

B

| | | |
|---|---|---|
| active ingredient | 2 | g |
| hexyl laurate | 5 | g |
| medium-chained triglyceride | 15 | g |
| ethanol | ad 100 | ml |

C.

| | | |
|---|---|---|
| active ingredient | 2 | g |
| oleyl oleate | 5 | g |
| N-methyl-pyrrolidone | 40 | g |
| isopropanol | ad 100 | ml |

The aqueous systems may also preferably be used for oral and/or intraruminal application. The compositions may also contain further additives, such as stabilisers, e.g. where appropriate epoxidised vegetable oils (epoxidised coconut oil, rapeseed oil, or soybean oil); antifoams, e.g. silicone oil, preservatives, viscosity regulators, binders, tackifiers, as well as fertilisers or other active ingredients to achieve special effects.

Further biologically active substances or additives, which are neutral towards the compounds of formula I and do not have a harmful effect on the host animal to be treated, as well as mineral salts or vitamins, may also be added to the described compositions.

The following examples serve merely to illustrate the invention without restricting it, the term active ingredient representing a substance listed in Examples or in Tables 1, 2 or 3.

### EXAMPLE 1

### 2-[1-(2,6-Dichloro-4-trifluoromethyl-phenyl)-1H-pyrrolo[3,2-b]pyridin-3-yl]-1,1,1-trifluoro-propan-2-ol

a) To a solution of 4-azaindole (400 mg) in dry THF (10 mL) cooled to −78° C. is added N-bromosuccinimide (783 mg) in one portion. The reaction mixture is stirred for 2 at −78° C. and allowed to warm up to room temperature. It is then poured on a 50 mL cartridge containing 7 g of Isolute® HM-N sorbent (column packed with diatomaceous earth support) and eluted with CH₂Cl₂ followed by acetone. The acetone fractions are evaporated under vacuum to give 4-aza-3-bromoindole as a white solid.

b) To a solution of 4-aza-3-bromoindole (680 mg) in dry DMF (7 mL) are added potassium carbonate (575 mg) and 3,5-dichloro-4-fluorobenzotrifluoride (630 μL). The reaction mixture is stirred for 22 h at room temperature and then poured on a 50 mL cartridge containing 7 g of Isolute® HM-N sorbent. Elution with CH₂Cl₂ gives 3-bromo-1-(2,6-dichloro-4-trifluoromethyl-phenyl)-1H-pyrrolo[3,2-b]pyridine as a yellow solid after removal of the solvent.

c) To a solution of 3-bromo-1-(2,6-dichloro-4-trifluoromethyl-phenyl)-1H-pyrrolo[3,2-b]pyridine (400 mg) in dry THF (5 mL) cooled to −78° C. is added a solution of n-butyl lithium (n-BuLi, 1.40 mL, 1.6 M in hexanes). The reaction mixture is stirred for 15 min. at −78° C., then trifluoroacetone (0.24 mL) is added dropwise and stirring is continued for a further 30 min. at −78° C. A saturated solution of NaHCO₃ is carefully added and the mixture is extracted with CH₂Cl₂. The combined organic phases are washed with brine, dried over MgSO₄ and filtered. After removal of the solvent the residue is purified by column-chromatography using an ethyl acetate/hexane gradient to give the title compound as a white solid as evaporation under vacuum.

### EXAMPLE 2

### Acetic acid 1-[1-(2,6-dichloro-4-trifluoromethyl-phenyl)-1H-pyrrolo[3,2-b]pyridin-3-yl]-2,2,2-trifluoro-1-methyl-ethyl ester

To a solution of (100 mg, synthesis according to Example 1) in dry DMF (1 mL) at 0° C. under nitrogen is added sodium hydride (7 mg, 95%). The reaction mixture is stirred for 15 min. at 0° C. and acetyl chloride (25 μL) is added. The mixture is stirred for 20 h at room temperature. After quenching with a saturated solution of NaHCO₃ and dichloromethane the mixture is filtered over a cartridge containing silica gel and ISOLUTE® HM-N. The cartridge containing ISOLUTE® HM-N is washed with dichloromethane. After removal of the solvent the residue is purified by preparative reverse phase chromatography on a Daisogel C18-ODS AP column with a water/formic acid (10,000:1) to acetonitrile/formic acid (10,000:1) gradient yielding the title compound after removal of the solvents.

### EXAMPLE 3

### 2-[6-(2,6-Dichloro-4-trifluoromethyl-phenyl)-6H-thieno[2,3-b]pyrrol-4-yl]-1,1,1-trifluoro-propan-2-ol

a) To a solution of 6H-thieno[2,3-b]pyrrole (250 mg) in dry THF (5 mL) cooled to 0° C. is added trifluoroacetic acid anhydride (0.37 mL). The mixture is allowed to warm to room temperature and stirred for 18 h. The volatiles are removed under vacuum and the residue is purified by column chromatography using an ethyl acetate/hexane gradient to give 2,2,2-trifluoro-1-(6H-thieno[2,3-b]pyrrol-4-yl)-ethanone as a beige solid.

b) To a solution of 2,2,2-trifluoro-1-(6H-thieno[2,3-b]pyrrol-4-yl)-ethanone (252 mg) in dry DMF (5 mL) are added dry potassium carbonate (158 mg) followed by 3,5-dichloro-4-fluorobenzotrifluoride (266 mg). The resulting suspension is stirred for 24 hours at 90° C. After removal of the solvent in vacuo the residue is partitioned between diethyl ether and water and the aqueous phase is extracted with diethyl ether. The combined organic phases are dried over MgSO₄, filtered and evaporated in vacuo. The residue is purified by column chromatography using an ethyl acetate/hexane gradient to give 1-[6-(2,6-dichloro-4-trifluoromethyl-phenyl)-6H-thieno[2,3-b]pyrrol-4-yl]-2,2,2-trifluoro-ethanone.

c) To a solution of 1-[6-(2,6-dichloro-4-trifluoromethyl-phenyl)-6H-thieno[2,3-b]pyrrol-4-yl]-2,2,2-trifluoro-ethanone (115 mg) in dry diethyl ether (5 mL) cooled to 0° C. is slowly added methyl magnesium bromide (0.18 mL, 3 M in diethyl ether). The mixture is stirred at 5° C. for 2 h and allowed to warm up to room temperature. The reaction mixture is then treated with saturated ammonium chloride solution and the organic layer separated. The aqueous phase is extracted with diethyl ether and the combined organic phases dried over magnesium sulphate and filtered. After evaporation of the solvent the residue is purified by preparative reverse phase chromatography on a Daisogel C18-ODS AP column with a water/formic acid (10,000:1) to acetonitrile/formic acid (10,000:1) gradient. The title compound is isolated by removal of the solvent.

The following compounds as outlined in Tables 1 and 2 are obtained using the methods as described in Examples 1 to 3 (MP=melting point). In the Tables the variables Z-1 to Z-12 have the following meaning

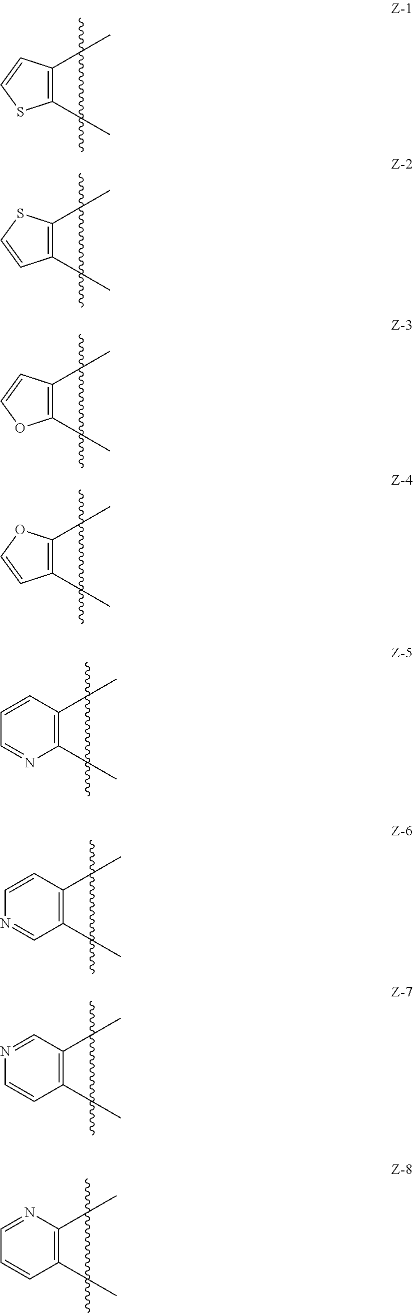

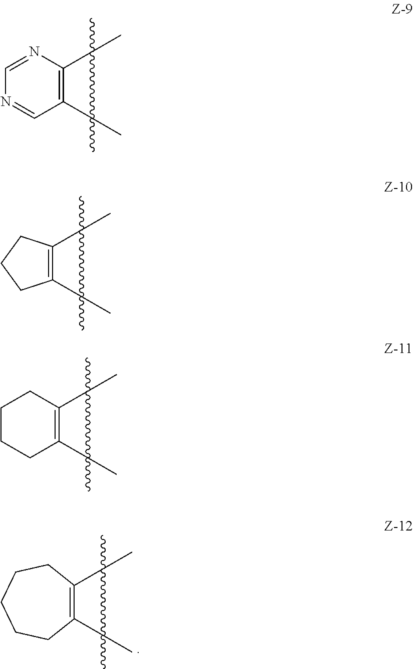

TABLE 1

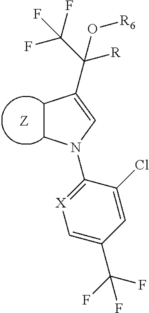

| Example | X | Z | R | R₆ | MP |
|---|---|---|---|---|---|
| 1.1 | CCl | Z-1 | Me | H | wax |
| 1.2 | CCl | Z-1 | CH═CH₂ | H | |
| 1.3 | CCl | Z-1 | C≡CH | H | |
| 1.4 | N | Z-1 | Me | H | |
| 1.5 | N | Z-1 | CH═CH₂ | H | |
| 1.6 | N | Z-1 | C≡CH | H | |
| 1.7 | CCl | Z-2 | Me | H | |
| 1.8 | CCl | Z-2 | CH═CH₂ | H | |
| 1.9 | CCl | Z-2 | C≡CH | H | |
| 1.10 | N | Z-2 | Me | H | |
| 1.11 | N | Z-2 | CH═CH₂ | H | |
| 1.12 | N | Z-2 | C≡CH | H | |
| 1.13 | CCl | Z-3 | Me | H | |
| 1.14 | CCl | Z-3 | CH═CH₂ | H | |
| 1.15 | CCl | Z-3 | C≡CH | H | |
| 1.16 | N | Z-3 | Me | H | |
| 1.17 | N | Z-3 | CH═CH₂ | H | |
| 1.18 | N | Z-3 | C≡CH | H | |
| 1.19 | CCl | Z-4 | Me | H | |
| 1.20 | CCl | Z-4 | CH═CH₂ | H | |
| 1.21 | CCl | Z-4 | C≡CH | H | |
| 1.22 | N | Z-4 | Me | H | |
| 1.23 | N | Z-4 | CH═CH₂ | H | |
| 1.24 | N | Z-4 | C≡CH | H | |
| 1.25 | CCl | Z-5 | Me | H | 189-190 |
| 1.26 | CCl | Z-5 | CH═CH₂ | H | |
| 1.27 | CCl | Z-5 | C≡CH | H | |
| 1.28 | N | Z-5 | Me | H | |
| 1.29 | N | Z-5 | CH═CH₂ | H | |
| 1.30 | N | Z-5 | C≡CH | H | |
| 1.31 | CCl | Z-6 | Me | H | |
| 1.32 | CCl | Z-6 | CH═CH₂ | H | |
| 1.33 | CCl | Z-6 | C≡CH | H | |
| 1.34 | N | Z-6 | Me | H | |
| 1.35 | N | Z-6 | CH═CH₂ | H | |
| 1.36 | N | Z-6 | C≡CH | H | |
| 1.37 | CCl | Z-7 | Me | H | 244-246 |
| 1.38 | CCl | Z-7 | CH═CH₂ | H | |
| 1.39 | CCl | Z-7 | C≡CH | H | |
| 1.40 | N | Z-7 | Me | H | |
| 1.41 | N | Z-7 | CH═CH₂ | H | |
| 1.42 | N | Z-7 | C≡CH | H | |
| 1.43 | CCl | Z-8 | Me | H | 187-190 |
| 1.44 | CCl | Z-8 | Et | H | |
| 1.45 | CCl | Z-8 | Me | COCH₃ | |
| 1.46 | CCl | Z-8 | Me | CO₂CH₃ | |
| 1.47 | CCl | Z-8 | Me | Me | |
| 1.48 | CCl | Z-8 | CH═CH₂ | H | |
| 1.49 | CCl | Z-8 | C≡CH | H | |
| 1.50 | N | Z-8 | Me | H | |
| 1.51 | N | Z-8 | CH═CH₂ | H | |
| 1.52 | N | Z-8 | C≡CH | H | |
| 1.53 | CCl | Z-9 | Me | H | |
| 1.54 | CCl | Z-9 | CH═CH₂ | H | |
| 1.55 | CCl | Z-9 | C≡CH | H | |
| 1.56 | N | Z-9 | Me | H | |
| 1.57 | N | Z-9 | CH═CH₂ | H | |
| 1.58 | N | Z-9 | C≡CH | H | |
| 1.59 | CCl | Z-10 | Me | H | |
| 1.60 | CCl | Z-10 | CH═CH₂ | H | |
| 1.61 | CCl | Z-10 | C≡CH | H | |
| 1.62 | N | Z-10 | Me | H | |
| 1.63 | N | Z-10 | CH═CH₂ | H | |
| 1.64 | N | Z-10 | C≡CH | H | |
| 1.65 | CCl | Z-11 | Me | H | 114-116 |
| 1.66 | CCl | Z-11 | CH═CH₂ | H | |
| 1.67 | CCl | Z-11 | C≡CH | H | |
| 1.68 | N | Z-11 | Me | H | |
| 1.69 | N | Z-11 | CH═CH₂ | H | |
| 1.70 | N | Z-11 | C≡CH | H | |
| 1.71 | CCl | Z-12 | Me | H | |
| 1.72 | CCl | Z-12 | CH═CH₂ | H | |
| 1.73 | CCl | Z-12 | C≡CH | H | |
| 1.74 | N | Z-12 | Me | H | |
| 1.75 | N | Z-12 | CH═CH₂ | H | |
| 1.76 | N | Z-12 | C≡CH | H | |

TABLE 2

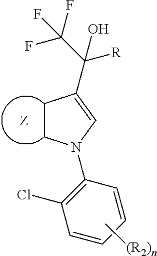

| Example | Z | R | (R₂)ₙ | MP |
|---|---|---|---|---|
| 2.1 | Z-1 | Me | 4-NO₂, 6-Cl | |
| 2.2 | Z-1 | Me | 4-OCF₃, 6-Cl | |
| 2.3 | Z-1 | Me | 4-Cl, 6-Cl | |
| 2.4 | Z-1 | Me | 4-Br, 6-Cl | |
| 2.5 | Z-1 | Me | 4-C≡CH, 6-Cl | |
| 2.6 | Z-1 | Me | 4-CN, 6-Cl | |
| 2.7 | Z-1 | Me | 4-CF₃, 6-NO₂ | |
| 2.8 | Z-1 | Me | 4-CF₃, 6-Br | |
| 2.9 | Z-1 | Me | 4-CF₃, 6-C≡CH | |
| 2.10 | Z-1 | Me | 4-CF₃, 6-CN | |
| 2.11 | Z-1 | Me | 4-SF₅, 6-Cl | |
| 2.12 | Z-8 | Me | 4-NO₂, 6-Cl | |
| 2.13 | Z-8 | Me | 4-OCF₃, 6-Cl | |
| 2.14 | Z-8 | Me | 4-Cl, 6-Cl | |
| 2.15 | Z-8 | Me | 4-Br, 6-Cl | |
| 2.16 | Z-8 | Me | 4-C≡CH, 6-Cl | |
| 2.17 | Z-8 | Me | 4-CN, 6-Cl | |
| 2.18 | Z-8 | Me | 4-CF₃, 6-NO₂ | |
| 2.19 | Z-8 | Me | 4-CF₃, 6-Br | |
| 2.20 | Z-8 | Me | 4-CF₃, 6-C≡CH | |
| 2.21 | Z-8 | Me | 4-CF₃, 6-CN | |
| 2.22 | Z-8 | Me | 4-SF₅, 6-Cl | |
| 2.23 | Z-8 | CH═CH₂ | 4-NO₂, 6-Cl | |
| 2.24 | Z-8 | CH═CH₂ | 4-OCF₃, 6-Cl | |
| 2.25 | Z-8 | CH═CH₂ | 4-Cl, 6-Cl | |
| 2.26 | Z-8 | CH═CH₂ | 4-Br, 6-Cl | |
| 2.27 | Z-8 | CH═CH₂ | 4-C≡CH, 6-Cl | |
| 2.28 | Z-8 | CH═CH₂ | 4-CN, 6-Cl | |
| 2.29 | Z-8 | CH═CH₂ | 4-CF₃, 6-NO₂ | |
| 2.30 | Z-8 | CH═CH₂ | 4-CF₃, 6-Br | |
| 2.31 | Z-8 | CH═CH₂ | 4-CF₃, 6-C≡CH | |
| 2.32 | Z-8 | CH═CH₂ | 4-CF₃, 6-CN | |
| 2.33 | Z-8 | CH═CH₂ | 4-SF₅, 6-Cl | |
| 2.34 | Z-8 | C≡CH | 4-NO₂, 6-Cl | |
| 2.35 | Z-8 | C≡CH | 4-OCF₃, 6-Cl | |
| 2.36 | Z-8 | C≡CH | 4-Cl, 6-Cl | |
| 2.37 | Z-8 | C≡CH | 4-Br, 6-Cl | |
| 2.38 | Z-8 | C≡CH | 4-C≡CH, 6-Cl | |
| 2.39 | Z-8 | C≡CH | 4-CN, 6-Cl | |
| 2.40 | Z-8 | C≡CH | 4-CF₃, 6-NO₂ | |
| 2.41 | Z-8 | C≡CH | 4-CF₃, 6-Br | |
| 2.42 | Z-8 | C≡CH | 4-CF₃, 6-C≡CH | |
| 2.43 | Z-8 | C≡CH | 4-CF₃, 6-CN | |
| 2.44 | Z-8 | C≡CH | 4-SF₅, 6-Cl | |
| 2.45 | Z-11 | Me | 4-NO₂, 6-Cl | |
| 2.46 | Z-11 | Me | 4-OCF₃, 6-Cl | |
| 2.47 | Z-11 | Me | 4-Cl, 6-Cl | |
| 2.48 | Z-11 | Me | 4-Br, 6-Cl | |
| 2.49 | Z-11 | Me | 4-C≡CH, 6-Cl | |
| 2.50 | Z-11 | Me | 4-CN, 6-Cl | |
| 2.51 | Z-11 | Me | 4-CF₃, 6-NO₂ | |
| 2.52 | Z-11 | Me | 4-CF₃, 6-Br | |
| 2.53 | Z-11 | Me | 4-CF₃, 6-C≡CH | |
| 2.54 | Z-11 | Me | 4-CF₃, 6-CN | |
| 2.55 | Z-11 | Me | 4-SF₅, 6-Cl | |

### Biological Examples:

1. Activity In Vitro Against Rhipicephalus sanguineus (Dog Tick).

A clean adult tick population is used to seed a suitably formatted 96-well plate containing the test substances to be evaluated for antiparasitic activity. Each compound is tested by serial dilution in order to determine its minimal effective dose (MED). Ticks are left in contact with the test compound for 10 minutes and are then incubated at 28° C. and 80% relative humidity for 7 days, during which the test compound's effect is monitored. Acaricidal activity is confirmed if adult ticks are dead.

In this test the compound number 1.43 showed more than 80% efficacy at 640 ppm.

2. Activity In Vitro Against Ctenocephalides felis (Cat Flea).

A mixed adult population of fleas is placed in a suitably formatted 96-well plate allowing fleas to access and feed on treated blood via an artificial feeding system. Each compound is tested by serial dilution in order to determine its MED. Fleas are fed on treated blood for 24 hours, after which the compound's effect is recorded. Insecticidal activity is determined on the basis of the number of dead fleas recovered from the feeding system.

In this test the compound No. 1.43 showed more than 80% efficacy at 100 ppm.

1. A compound of formula I 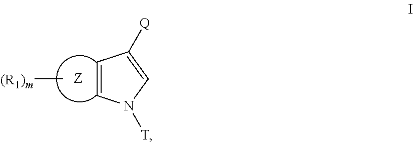
wherein:
Z is an annulated carbocyclic or heterocyclic ring with the exception of a phenyl ring;
R₁ is halogen, cyano, nitro, C₁-C₆-alkyl, C₃-C₆-cycloalkyl, C₂-C₆-alkenyl, C₂-C₆-alkynyl, hydroxyl-C₁-C₆-alkyl, halo-C₁-C₆-alkyl, hydroxy, C₁-C₆-alkoxy, halo-C₁-C₆-alkoxy, SH, C₁-C₆-alkylthio, halo-C₁-C₆-alkylthio, C₁-C₆-alkylsulfinyl, halo-C₁-C₆-alkylsulfinyl, C₁-C₆-alkylsulfonyl, halo-C₁-C₆-alkylsulfonyl, SO₃R₃, SO₂NR₃R₄, NR₃R₄, COR₃, COOR₃, or CONR₃R₄,
whereby, if m is greater than 1, the meaning of each R₁ may be identical or different;
T is a group of the formula II 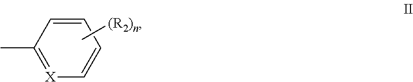
wherein:
R₂ is halogen, cyano, nitro, C₁-C₆-alkyl, halo-C₁-C₆-alkyl, C₃-C₆-cycloalkyl, C₂-C₆-alkenyl, C₂-C₆-alkynyl, hydroxy, C₁-C₆-alkoxy, halo-C₁-C₆-alkoxy, SH, C₁-C₆-alkylthio, halo-C₁-C₆-alkylthio, C₁-C₆-alkylsulfinyl, halo-C₁-C₆-alkylsulfinyl, C₁-C₆-alkylsulfonyl, halo-C₁-C₆-alkylsulfonyl, SO₃R₃, SO₂NR₃R₄, NR₃R₄, COR₃, COOR₃, CONR₃R₄, or SF₅,
whereby, if n is greater than 1, the meaning of each R₂ may be identical or different;
each of R₃ and R₄ is independently:
hydrogen;
C₁-C₆-alkyl, optionally substituted with halogen, cyano, NO₂, C₁-C₄-alkoxy, C₁-C₄-alkylcarbonyl, C₁-C₄-alkylcarbonyloxy, or C₁-C₄-alkoxycarbonyl; or
C₁-C₂-alkoxyC₁-C₂-alkyl;
m signifies 0, 1, 2, or 3;
n signifies 1, 2, 3, or 4;
X is N or C(R₂′), wherein R₂′ is hydrogen halogen, cyano, nitro, C₁-C₆-alkyl, halo-C₁-C₆-alkyl, C₃-C₆-cycloalkyl, C₂-C₆-alkenyl, C₂-C₆-alkynyl, hydroxy, C₁-C₆-alkoxy, halo-C₁-C₆-alkoxy, SH, C₁-C₆-alkylthio, halo-C₁-C₆-alkylthio, C₁-C₆-alkylsulfinyl, halo-C₁-C₆-alkylsulfinyl, C₁-C₆-alkylsulfonyl, halo-C₁-C₆-alkylsulfonyl, SO₃R₃ SO₂NR₃R₄ NR₃R₄ COR₃ COOR₃ CONR₃R₄ or SF₅;
with the proviso that R₂′ is not hydrogen if n is 1;
Q is a group of the formula III 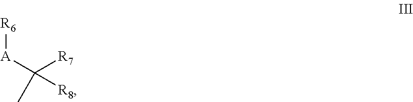
wherein:
A is O, S, S(O), or S(O₂);
R₆ is hydrogen,
C₁-C₆-alkyl,
C₁-C₆-hydroxyalkyl,
C₃-C₆-cycloalkyl,
C₃-C₆-cycloalkylmethyl,
C₁-C₄-alkoxymethyl,
C₁-C₂-alkoxy-C₁-C₂-alkoxymethyl,
phenoxymethyl, optionally substituted in the phenyl moiety by halogen, C₁-C₂-alkyl, halo-C₁-C₂-alkyl or C₁-C₂-alkoxy,
benzyloxymethyl,
C₂-C₆-alkenyl,
C₂-C₆-alkynyl,
halo-C₁-C₆-alkyl,
halo-C₃-C₆-cycloalkyl,
halo-C₁-C₆-cycloalkylmethyl,
halo-C₂-C₆-alkenyl,
halo-C₂-C₆-alkynyl,
COR₃,
COOR₃,
CONR₃R₄,
CSNR₃R₄,
C₁-C₄-alkyl-silyl,
phenyl, optionally substituted by halogen, nitro, cyano, C₁-C₄-alkyl, halo-C₁-C₄-alkyl, C₁-C₄-alkoxy, halo-C₁-C₄-alkoxy, NH₂, N—C₁-C₄-alkylamino, N,N-di-C₁-C₄-alkylamino, C₁-C₄--alkylthio COR₃, COOR₃, or CONR₃R₄, or
phenyl-C₁-C₂-alkyl, optionally substituted in the phenyl moiety by halogen, nitro, cyano, hydroxy, C₁-C₄-alkyl, halo-C₁-C₄-alkyl, C₁-C₄-alkoxy, halo-C₁-C₄-alkoxy, NH₂, N—C₁-C₄-alkylamino, N,N-di-C₁-C₄-alkylamino, C₁-C₄-alkylthio, COR₃, COOR₃, or CONR₃R₄;
R₇ is C₁-C₆-alkyl, C₂-C₆-alkenyl, C₂-C₆-alkynyl, halo-C₁-C₆-alkyl, halo-C₂-C₆-alkenyl, halo-C₂-C₆-alkynyl, C₃-C₈-cycloalkyl, halo-C₃-C₈-cycloalkyl, hydroxy-C₁-C₆-alkyl, COR₃ or COOR₃; and
R₈ is hydrogen;
C₁-C₆-alkyl optionally substituted by halogen, C₁-C₄-alkylthio, hydroxyl, C₁-C₄-alkoxy, amino or N-mono- or N,N-di-C₁-C₄-alkyl;
C₂-C₆-alkenyl, optionally substituted with halogen, hydroxyl, or C₁-C₂-alkoxy;
C₂-C₆-alkynyl, optionally substituted with halogen, hydroxyl, or C₁-C₂-alkoxy;
C₃-C₈-cycloalkyl, optionally substituted with halogen, hydroxyl, or C₁-C₂-alkoxy;
C₅-C₆-cycloalkylmethyl, wherein 1 to 3 carbon atoms of the cycloalkyl moiety are optionally replaced with a heteroatom selected from the group consisting of NH, N(C₁-C₄-alkyl), O, and S;
benzyl, optionally substituted with halogen, hydroxyl or C₁-C₂-alkoxy;
phenyl, optionally substituted with halogen, halo-C₁-C₂-alkyl, or halo-C₁-C₂-alkoxy;
cyano;
COR₃; or
COOR₃; or
R₇ and R₈ together with the carbon atoms to which they are attached, form an aliphatic ring of 3 to 6 atoms, optionally including a heteroatom selected from the group consisting of nitrogen, sulfur, oxygen, and carbonyl, and optionally substituted with 1 to 4 substituents independently selected from the group consisting of halogen, CN, NO₂, hydroxy, C₁-C₄-alkyl, and C₁-C₄-alkoxy.

2. A compound according to claim 1 of formula Ia 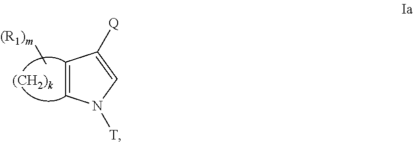
wherein k is 3, 4, or 5; and
R₁, Q, T, and m are as defined.

3. A compound according to claim 1 of formula Ib 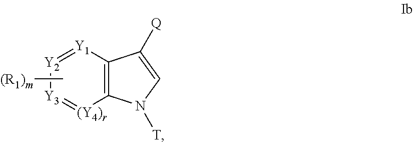
wherein 1 or 2 of the radicals Y₁, Y₂, Y₃, and Y₄ are independently a heteroatom selected from the group consisting of N, O, and S;
the remaining radicals are CH;
r is 0 or 1; and
R₁, Q, T, and m are as defined.

4. A compound according to claim 1, wherein T is a radical of formula IIa 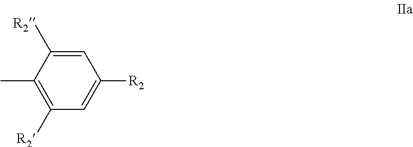
wherein R₂′ and R₂″ are each halogen and R₂ is halo-C₁-C₂-alkyl.

5. A compound according to claim 1, wherein Q is a radical of formula III, 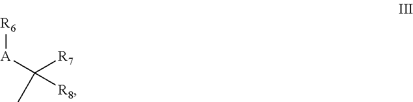
wherein:
A is O;
R₆ is hydrogen;
R₇ is CF₃; and
R₈ is hydrogen;
C₁-C₄-alkyl, optionally substituted with halogen, hydroxyl or C₁-C₂-alkoxy;
C₂-C₄-alkenyl, optionally substituted with halogen, hydroxyl or C₁-C₂-alkoxy;
C₂-C₄-alkynyl, optionally substituted with halogen, hydroxyl or C₁-C₂-alkoxy;
benzyl, optionally substituted with halogen, hydroxyl or C₁-C₂-alkoxy; or
phenyl, optionally substituted with halogen-, halo-C₁-C₂-alkyl- or halo-C₁-C₂-alkoxy.

6. A compound of formula I according to claim 1, 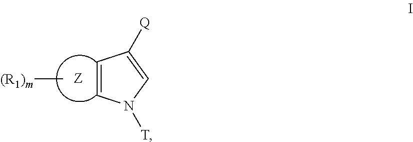
wherein:
R₁ is halogen or C₁-C₄-alkyl;
m signifies 0, 1, or 2;
T is a radical of formula II, 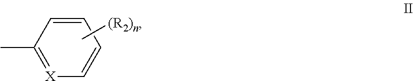
wherein:
X is N or C(R₂′);
n is 1 or 2; and
R₂ and R₂′ are each independently halogen, cyano, nitro, C₂-C₄-alkynyl, halo-C₁-C₄-alkyl halo-C₁-C₄-alkoxy, or SF₅,
whereby, if n is 2, the meanings of R₂ may be identical or different;
Q is a group of formula III, 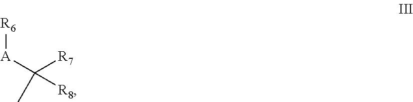
wherein:
A is O;
R₆ is hydrogen, C₁-C₄-alkyl, or C(O)—C₁-C₂-alkyl;
R₇ is C₁-C₄-alkyl or halo-C₁-C₄-alkyl; and
R₈ is hydrogen; C₁-C₄-alkyl, halo-C₁-C₄-alkyl, C₂-C₄-alkenyl, or C₂-C₄-alkynyl; and
Z is as defined.

7. A compound according to claim 1 of the formula Ic 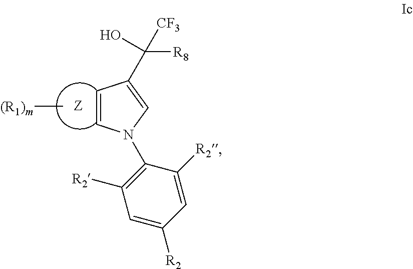
wherein:
R₁ is halogen or C₁-C₂-alkyl;
m is 0 or 1;
R₂ is trifluoromethyl;
R₂′ and R₂″ are each chlorine;
R₈ is C₁-C₂-alkyl, halo-C₁-C₂-alkyl, C₂-C₄-alkenyl, or C₂-C₄-alkynyl; and
the ring Z is a 5- or 6-membered cycloaliphatic ring or a 5- or 6-membered heteroaromatic ring having one heteroatom selected from the group consisting of N, Q and S.

8. A process for the preparation of a compound of the formula I according to claim 1, comprising the steps of
(i) halogenating a compound of formula IV 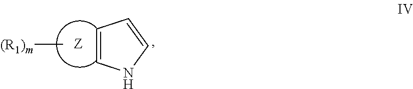
wherein R₁, Z, and m are as defined, to yield a compound of the formula IVa 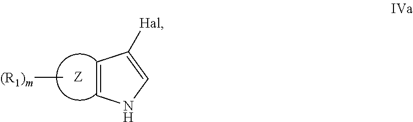
wherein Hal is halogen, and R₁, Z, and m are as defined;
(ii) reacting the compound of the formula IVa obtained according to step (i) with a compound of the formula V
L-T V,
wherein T is as defined and L is a leaving group, optionally in the presence of a basic catalyst, to yield a compound of the formula IVb 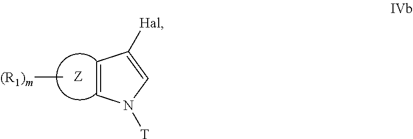
wherein Hal is halogen, and R₁, T, Z and m are as defined; and
(iii) reacting the compound of the formula IVb with a lithium-organic compound, followed by reacting the resulting lithium-organic compound with a ketone of the formula VI 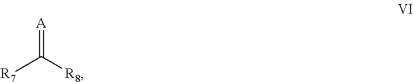
wherein A, R₇, and R₈ are as defined, to yield a compound of the formula I.

9. A method of controlling parasites in or on warm-blooded animals, excluding humans, and on plants, comprising administering an effective amount of a compound of formula I according to claim 1 to the parasites or their locus.

10. A composition for the control of parasites in or on warm-blooded animals, excluding humans, which contains as active ingredient an effective amount of a compound of formula I according to claim 1, in addition to a physiologically acceptable carrier or dispersant.

11. A method of controlling parasites in or on warm-blooded animals, excluding humans, and on plants, comprising administering an effective amount of a compound of formula I according to claim 7 to the parasites or their locus.

12. (canceled)

13. A compound according to claim 4, wherein T is a radical of the formula IIa 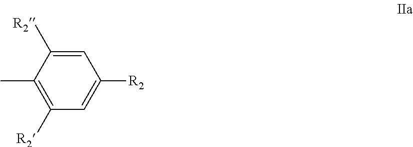
wherein R₂′ and R₂″ are each chlorine and R₂ is halo-C₁-C₂-alkyl.

14. A compound according to claim 1 of the formula Ic 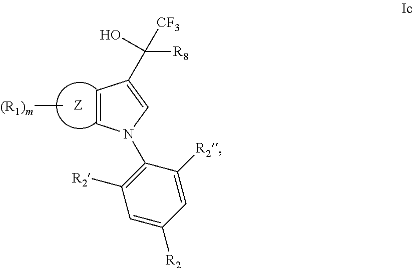
wherein
R₁ is halogen or C₁-C₂-alkyl;
m is 0;
R₂ is trifluoromethyl;
each of R₂′ and R₂″ are chlorine;
R₈ is C₁-C₂-alkyl, halo-C₁-C₂-alkyl, C₂-C₄-alkenyl, or C₂-C₄-alkynyl; and
the ring Z is a 5- or 6-membered cycloaliphatic ring or a 5- or 6-membered heteroaromatic ring having one heteroatom selected from the group consisting of N, O, and S.

16. A compound according to claim 5, wherein R₈ is hydrogen, C₁-C₄-alkyl, halo-C₁-C₄-alkyl, C₂-C₄-alkenyl, or C₂-C₄-alkynyl.

17. A compound according to claim 5, wherein R₈ is hydrogen, C₁-C₂-alkyl, CF₃, ethenyl, or ethynyl.
